(19) United States
(12) Patent Application Publication
(10) Pub. No.: US 2015/0346221 A1
(43) Pub. Date: Dec. 3, 2015

(54) QUANTIFICATION METHOD FOR SULFUR-CONTAINING ORGANIC COMPOUND

(71) Applicant: KOREA RESEARCH INSTITUTE OF STANDARDS AND SCIENCE, Yuseong-gu Daejeon (KR)

(72) Inventors: Yong Hyeon Yim, Daejeon (KR); Hyun Seok Lee, Daejeon (KR); Sook Heun Kim, Daejeon (KR); Kyoung Seok Lee, Daejeon (KR); Ji Seon Jeong, Daejeon (KR); Sook-Kyoung Kim, Daejeon (KR)

(21) Appl. No.: 14/653,798

(22) PCT Filed: Dec. 20, 2013
(86) PCT No.: PCT/KR2013/011971

§ 371 Date: Jun. 18, 2015

(30) Foreign Application Priority Data
Dec. 20, 2012 (KR) 10-2012-0150109
Dec. 20, 2013 (KR) 10-2013-0159867

(51) Int. Cl. G01N 33/74; G01N 33/68

(52) U.S. Cl. CPC G01N 33/74; G01N 33/6815

(57) ABSTRACT

Provided is a quantification method for a sulfur-containing organic compound. The quantification method may quantify the sulfur-containing organic compound without limitation. Preferably, the quantification method is more effective for quantifying organic compounds of which quantification is difficult to perform, such as a macromolecule such as a biomaterial, or the organic compound having a hygroscopic property or being present in a hydrated form, and significantly effectively quantifying high purity standard materials of peptide or protein among the biomaterials.

### TECHNICAL FIELD

The present invention relates to a quantification method for a sulfur-containing organic compound.

### BACKGROUND ART

The present invention relates to a quantification method for a sulfur-containing organic compound. In general, accuracy and traceability of quantitative analysis have significantly important meaning as a measure to determine reliability on experimental results and correlating possibility between experiments. In particular, when securing high purity standard material having measurement traceability in quantitative analysis for an organic compound and a biomaterial is not preceded, reliability of measurement may be seriously affected. As a quantitative value of the high purity standard material or standard solution, a manufacturing value obtained by manufacturing the material or solution by a weighting method based on various purity analysis results of the corresponding materials, and correcting the purity is used. As another method, in a case of complex materials such as a biomolecule, there is a method in which the high purity standard material in an organic compound level such as nucleic acid monomer/amino acid, and the like, that is a basic unit of the biomolecule, is secured by the above-described method, and a characteristic value or the quantitative value of a high purity biomaterial is obtained based on the secured high purity standard material. For example, to quantify a protein standard material/standard solution, a method for enzymatically decomposing protein into a peptide unit, and quantifying the peptide has been largely used. However, this method requires a peptide standard material having accurate content. In a case of high purity standard material of peptide or protein, a method for determining a peptide content or a protein content by quantifying amino acids produced by hydrolysis of the peptide or the protein has been widely utilized (J. Chromatography A, 1218, 6596-6602 (2011)). However, in order to use this method, a standard material of the amino acid to be used for quantification needs to be secured, and a hydrolysis condition needs to be established so as to secure that an efficiency in a process in which protein/peptide is hydrolyzed into an amino acid is close to 100%. As an example of a technology for securing the quantification method for a biomaterial, a method for cutting DNA into nucleic acid monomers by an enzyme and quantifying these monomers by Isotope dilution HPLC-MS to quantify DNA has been attempted (O'Connor, G., et. al., Anal. Chem., 74, 3670-3676 (2002)). In addition, in a case of protein, UV absorbance measurement, a Biuret method, a BCA method, a Lowry method, a Bradford method, or the like, has been mainly utilized to quantify protein; however, problems in view of sensitivity and dynamic range still have not been solved. Further, a mass of organic compound being present in a hydrated form or having a hygroscopic property varies according to the number of water molecules included in a sample, such that it is significantly difficult to measure an accurate mass.

### DISCLOSURE

### Technical Problem

An object of the present invention is to provide a quantification method for a sulfur-containing organic compound. More specifically, there is provided a quantification method of a biomaterial selected from the group consisting of an organic compound containing sulfur, peptide or protein including methionine, cysteine, or both of methionine and cysteine, and DNA, RNA or PNA which is modified so as to contain sulfur. In particular, when the organic compound or the biomaterial is present in a hydrated form or has a hygroscopic property, the quantification method may effectively solve a problem in that reliability of manufacturing values obtained by a weighting method from a high purity organic compound or a high purity standard material of which purity is confirmed becomes deteriorated in the existing methods.

### Technical Solution

In one general aspect, there is provided a quantification method for a sulfur-containing organic compound, using a sulfur isotope ratio. More specifically, the quantification method for a sulfur-containing organic compound may include steps (1) to (4) below:

(1) preparing a sample blend so that a theoretical isotopic ratio between any one isotope selected from ³³S, ³⁴S, and ³⁶S and a base isotope (³²S) having the largest natural abundance ratio is 0.2 to 5 in a sample solution, by diluting a sample solution including the sulfur-containing organic compound with an internal standard solution in which any one isotope selected from ³³S, ³⁴S, and ³⁶S is concentrated;

(2) preparing a calibration blend so as to have the same theoretical isotopic ratio as the sample blend prepared in step (1), by mixing the internal standard solution in which any one isotope selected from ³³S, ³⁴S, and ³⁶S is concentrated with a sulfur standard solution having a natural abundance ratio;

(3) recovering an inorganic form of sulfate, by decomposing the sulfur-containing organic compound from the sample blend prepared in step (1); and

(4) measuring an isotope ratio between any one isotope selected from ³³S, ³⁴S, and ³⁶S and isotope ³²S of the sulfate recovered in step (3), and measuring an isotope ratio between any one isotope selected from ³³S, ³⁴S, and ³⁶S and isotope ³²S of the calibration blend prepared in step (2).

The sulfur-containing organic compound may be protein or peptide including methionine, cysteine, or both of methionine and cysteine as well as an organic compound such as methionine, cysteine, or the like.

In step 3), the sample blend including the sulfur-containing organic compound may be preferably treated by simultaneously performing electromagnetic wave irradiation and acid decomposition to be decomposed into the inorganic form of sulfate, but is not limited thereto. The simultaneously performing of the electromagnetic wave irradiation and acid decomposition may include putting the sample blend into an electromagnetic wave oven to perform acid decomposition while maintaining a temperature at 100° C. to 300° C. for 10 minutes to 240 minutes, and repeating the electromagnetic wave irradiation and acid decomposition to perform electromagnetic wave treatment. The simultaneously performing of the electromagnetic wave irradiation and acid decomposition is a method for effectively decomposing sulfur into an inorganic element form, but is not limited thereto, and sulfur may be decomposed into an inorganic form under various conditions. For acid decomposition, an oxidizing agent having a sufficient amount for converting all constitutional elements of the organic compound into an inorganic element form needs to be added. When it is assumed that the total number of moles of carbon, hydrogen, nitrogen, and sulfur atoms constituting the sulfur-containing organic compound is 1, the oxidizing agent may be preferably have a molar ratio of at least 10 times or more. The oxidizing agent may be used without limitation except for sulfuric acid, and preferable examples of the oxidizing agent may include nitric acid, perchloric acid, hydrogen peroxide, or a mixture thereof.

When the oxidizing agent having a molar ratio less than 10 times is added, acid decomposition may not be sufficiently generated, and when the oxidizing agent having a molar ratio more than 10 times is added, there is no serious problems; however, acid concentration of a final analysis solution is no more than 10 wt % to be appropriate for ICP/MS analysis.

The isotope ratio between any one isotope selected from ³³S, ³⁴S, and ³⁶S and isotope ³²S of the sample blend, and the isotope ratio between any one isotope selected from ³³S, ³⁴S, and ³⁶S and isotope ³²S of the calibration blend, obtained in step (4) may be applied to Equation 1 below to obtain concentration of the sample solution to be measured:

$${C}_{x} = \{ {C}_{z} \cdot \frac{{m}_{z} {m}_{y}}{{wm}_{z} {m}_{y}} \cdot \frac{{R}_{y} - {R}_{b}}{{R}_{b} - {R}_{x}} \cdot \frac{{R}_{b} - {R}_{z}}{{R}_{y} - {R}_{b}} \cdot \frac{{\sum }_{i} {R}_{xi}}{{\sum }_{i} {R}_{zi}} \} - ( {C}_{blank} ) \qquad 〈 Equation 1 〉$$

Cₓ: Concentration of sample solution (x) to be measured,

mₓ: Mass of sample solution (x) measured by using a scale,

m_{y}: Mass of internal standard solution (y) in which any one isotope selected from ³³S, ³⁴S, and ³⁶S added to sample solution (x) is concentrated,

m_{y}′: Mass of internal standard solution (y) in which any one isotope selected from ³³S, ³⁴S, and ³⁶S added to sulfur standard solution (z) for calibration is concentrated,

C_{z}: Concentration of sulfur standard solution (z) for calibration,

Rₓ: Sulfur isotope ratio (any one isotope selected from ³³S, ³⁴S, and ³⁶S/³²S isotope) of sample solution (x),

R_{y}: Sulfur isotope ratio (any one isotope selected from ³³S, ³⁴S, and ³⁶S/³²S isotope) of an internal standard solution (y) in which any one isotope selected from ³³S, ³⁴S, and ³⁶S is concentrated,

R_{z}: Sulfur isotope ratio (any one isotope selected from ³³S, ³⁴S, and ³⁶S/³²S isotope) of sulfur standard solution (z)

Rₓᵢ: Sulfur i-th isotope ratio (³²S/³²S, ³³S/³²S, ³⁴S/³²S, ³⁶S/³²S) of sample solution (x),

R_{zi}: Sulfur i-th isotope ratio (³²S/³²S, ³³S/³²S, ³⁴S/³²S or ³⁶S/³²S) of sulfur standard solution (z) for calibration,

R_{b}: Sulfur isotope ratio (any one isotope selected from ³³S, ³⁴S, and ³⁶S/³²S isotope) of sample blend,

R_{b′}: Sulfur isotope ratio (any one isotope selected from ³³S, ³⁴5, and ³⁶S/³²S isotope) of calibration blend,

w: Dry mass calibration factor, and

C_{blank}: Concentration of blank sample.

### Advantageous Effects

The present invention provides a quantification method for sulfur-containing organic compound. More specifically, the present invention provides the quantification method of an organic compound by adding an inorganic form of sulfur solution in which one of sulfur isotopes (³³5, ³⁴S and ³⁶S) is concentrated as an internal standard material to an organic compound to be quantified to thereby prepare a sample blend, adding the same internal standard material to a sulfur elemental standard solution to thereby prepare a calibration blend, and measuring and comparing a sulfur isotope ratio between the sample blend and the calibration blend. The quantification method according to the present invention may be significantly effectively used, not only for quantification of the biomaterial which is a macromolecule, particularly, peptide or protein including methionine or eystcine but also for quantification of an organic compound having a strong hygroscopic property or being present in a hydrated form that is difficult to perform an accurate quantification.

In particular, the quantification method according to the present invention may provide quantification results with high reliability and consistency on the high purity first standard material such as an organic compound, protein/peptide, or the like, to thereby be utilized for analyzing properties of the high purity standard solution and establishing measurement standard. The standard solution as prepared above may be used as a standard solution for calibration for quantitative analysis of various organic compounds and biomolecules to be utilized for securing reliability on quantification results.

### DESCRIPTION OF DRAWINGS

FIG. 1A is a schematic view showing a sample blend (b) consisting of a sample solution (x) having a sulfur isotopic natural abundance ratio (³⁴S/³²S) and ³⁴S highly concentrated internal standard solution (y), and FIG. 1B is schematic view showing a calibration blend (b′) consisting of a sulfur element standard solution (z) and ³⁴S highly concentrated internal standard solution (y).

FIG. 2 is a conceptual diagram showing a quantification method for a sulfur-containing organic compound by using an isotope dilution inductively coupled plasma mass spectrometry (ICP/MIS).

FIG. 3 is a graph showing a relationship between an error magnification factor (EMF) and an isotope ratio between the sample blend and the calibration blend in sulfur isotope dilution. R indicates a sulfur isotope ratio (³⁴S/³²S).

FIG. 4 shows quantification results of human growth hormone (hGH) based on sulfur isotope measurements.

FIG. 4A shows quantification results of human growth hormone prepared in a first batch, FIG. 4B shows comparison results between sulfur isotope-based quantification and amino acid-based quantification, wherein I, F, P, and V indicate results obtained by hydrolyzing proteins to be amino acids and quantifying isoleucine, plienylalanine, proline, and valine to calculate protein content

FIG. 5 is a SEC-UV chromatogram of human growth hormone. A main peak at 3.3 minutes indicates the human growth hormone, and a small peak at 4.1 minutes indicates a small molecule.

FIG. 6 shows results obtained by monitoring only m/z 32 and m/z 34 corresponding to sulfur elements, among components separated by SEC for analysis of sulfur-containing impurities in a small amount included in a solvent, using ICP/MS. The peak of the sulfur-containing small molecule impurities next to the peak of the human growth hormone molecule at 3.3 minutes is not shown.

FIG. 7 shows sulfur isotope-based quantification results and amino acid-based quantification results of the human growth hormone prepared in a second batch.

FIG. 8 shows sulfur isotope-based quantification results and amino acid-based quantification results of hGH T2 peptide.

FIG. 9 shows sulfur isotope-based quantification results and amino acid-based quantification results of hGI-i T11 peptide.

FIG. 10 shows sulfur isotope-based quantification results of methionine.

FIG. 11 shows sulfur isotope-based quantification results of a sample NIST SRM 2389a according to a sample pre-treatment method.

FIG. 12 shows evaluation results of homogeneity of human growth hormone prepared in the second batch.

FIG. 13 shows sulfur isotope-based certified results and amino acid-based or peptide-based quantification results of the human growth hormone prepared in the second batch.

### BEST MODE

The present invention relates to a quantification method for a sulfur-containing organic compound, using a sulfur isotope ratio. More specifically, the present invention relates to the quantification method for measuring an isotope ratio of sulfur contained in the organic compound and calculating an amount of the sulfur-containing organic compound from the measured sulfur isotope ratio. The method for measuring the isotope ratio of sulfur contained in the organic compound is preferably performed by inductively coupled plasma mass spectrometry (ICP/MS), but the present invention is not limited thereto.

It is known that sulfur present in nature has 4 isotopes including ³²S, ³³S, ³⁴S, and ³⁶S, among them, ³²S mostly occupies by 95.02% and ³⁴S occupies by 4.21%. The present invention is characterized by using an isotope ratio between any one isotope selected from ³³S, ³⁴S, and ³⁶S isotopes contained in an organic compound in a sample solution (x) and ³²S isotope, and more specifically, by using an isotope ratio (R_{b}) between any one isotope selected from ³³S, ³⁴S, and ³⁶S of a sample blend (b) and ³²S isotope, wherein the sample blend is diluted by adding an internal standard solution (y) in which any one isotope selected from ³³S, ³⁴S, and ³⁶S is concentrated to the sample solution, and by using a corresponding isotope ratio (R_{b′}) of a calibration blend (b′) consisting of a sulfur standard solution (z) and the same internal standard solution (y) as above.

According to an exemplary embodiment of the present invention, the quantification method for a sulfur-containing organic compound, using a sulfur isotope ratio (a ratio between any one isotope selected from ³³S, ³⁴S, and ³⁶S and ³²S isotope) may include:

(1) preparing the sample blend (b) so that a theoretical isotopic ratio (any one isotope selected from ³³S, ³⁴S, and ³⁶S/³²S isotope) is 1, by diluting the sample solution (x) including a sulfur-containing organic compound with an internal standard solution (y) in which any one isotope selected from ³³S, ³⁴S, and ³⁶S is concentrated;

(2) preparing a calibration blend (b′) so that a theoretical isotopic ratio (any one isotope selected from ³³S, ³⁴S, and ³⁶S/³²S isotope) is 0.2 to 5, by mixing the internal standard solution (y) in which any one isotope selected from ³³S, ³⁴S, and ³⁶S is concentrated with a sulfur standard solution (z) having a natural abundance ratio as a sulfur isotope ratio, wherein the step (1) and the step (2) are simultaneously performed;

(3) decomposing sulfur in the sulfur-containing organic compound in step (1) into an inorganic form of sulfate and recovering the inorganic form of sulfate; and

(4) measuring an isotope ratio between any one isotope selected from ³³S, ³⁴S, and ³⁶S of the sample blend recovered in step (3) and ³²S, and measuring an isotope ratio between any one isotope selected from ³³S, ³⁴S, and ³⁶S of the calibration blend prepared in step (2) and ³²S.

In the quantification method according to the present invention, the sulfur-containing organic compound may be quantified without limitation; however, it is preferable to quantify organic compounds or biomaterials having a high hygroscopic property or being present in a hydrated form which have high possibility of deflection for quantification by a general chemical scale, mass spectrometry, or other analysis equipments. Preferable biomaterial may include peptide, protein, or DNA, RNA, or PNA which is modified so as to contain sulfur, and the like, but is not limited thereto. The peptide and the protein refer to peptide or protein including methionine, cysteine, or both of methionine and cysteine. The protein mainly consists of carbon, hydrogen, and oxygen, and contains sulfur at a low ratio. Besides, the protein includes heteroatom such as phosphorus, but the protein including heteroatom is shown only in a specific protein as a modified form. The carbon, hydrogen, and oxygen are elements that are relatively highly present in composition of a protein, that is, they are elements having a relatively high abundance ratio; however, there are many cases that the carbon, hydrogen, and oxygen elements are present in the air or even in a solvent used for measurement, such that it may be difficult to control a background signal. Therefore, an absolute quantification method of protein, based on measurement for sulfur, which is present as methionine or cysteine in most of protein, while having a small abundance ratio is preferable.

In step (3) above, the decomposing of sulfur in the sulfur-containing organic compound into an inorganic form of sulfate is performed by simultaneously performing electromagnetic wave irradiation and acid decomposition on the sample blend (b) so that the sulfur in the sulfur-containing organic compound is decomposed into a sulfate which is an element form, more specifically, by putting an acid-treated sample blend (b) into an electromagnetic wave oven to perform acid decomposition while maintaining a temperature at 100° C. to 300° C. for 10 minutes to 240 minutes, and repeating the electromagnetic wave irradiation and acid decomposition to perform electromagnetic wave treatment. The simultaneously performing of the electromagnetic wave irradiation and acid decomposition is a method for effectively decomposing sulfur into an inorganic element form, but is not limited thereto, and sulfur may be decomposed into an inorganic form under various conditions. In addition, the acid usable for the acid decomposition may be preferably nitric acid, and perchloric acid, but may be used without limitation except for sulfuric acid. In addition, the acid decomposition may be performed by further including hydrogen peroxide (H2O2) in order to increase oxygenative power.

When it is assumed that the total number of moles of carbon, hydrogen, nitrogen, and sulfur atoms of the organic compound to be decomposed is 1, the oxidizing agent may preferably have a molar ratio at least 10 times or more. When the oxidizing agent having a molar ratio less than 10 times is added, acid decomposition may not be sufficiently generated, and when the oxidizing agent having a molar ratio more than 10 times is added, there is no serious problems; however, acid concentration of a final analysis solution is preferably no more than 10 wt % to be appropriate for ICP/MS analysis.

When the isotope ratio between any one isotope selected from ³³S, ³⁴S, and ³⁶S of the sample blend and ³²S, and the isotope ratio between any one isotope selected from ³³S, ³⁴S, and ³⁶S of the calibration blend and ³²S, obtained in step (4), are obtained, a protein content converted by the sulfur content may be calculated by Equation 1 below.

In Equation 1 below, Rₓ and R_{z} may be values obtained according to a natural abundance ratio of sulfur provided in IUPAC, 0.042/0.9501(³⁴S/³²S), or by direct measurement using a mass spectrometer for isotope ratio measurement.

$${C}_{x} = \{ {C}_{z} \cdot \frac{{m}_{z} {m}_{y}}{{wm}_{z} {m}_{y}} \cdot \frac{{R}_{y} - {R}_{b}}{{R}_{b} - {R}_{x}} \cdot \frac{{R}_{b} - {R}_{z}}{{R}_{y} - {R}_{b}} \cdot \frac{{\sum }_{i} {R}_{xi}}{{\sum }_{i} {R}_{zi}} \} - ( {C}_{blank} ) \qquad 〈 Equation 1 〉$$

Cₓ: Concentration of sample solution (x) to be measured,

mₓ: Mass of sample or sample solution (x) collected by using a scale,

m_{y}: Mass of internal standard solution (y) in which any one isotope selected from ³³S, ³⁴S, and ³⁶S added to the sample solution (x) is concentrated,

m_{y′}: Mass of internal standard solution (y) in which any one isotope selected from ³³S, ³⁴S, and ³⁶S added to sulfur standard solution (z) for calibration is concentrated,

C_{z}: Concentration of sulfur standard solution (z) for calibration,

Rₓ: Sulfur isotope ratio (any one isotope selected from ³³S, ³⁴S, and ³⁶S/³²S isotope) of sample solution (x),

R_{y}: Sulfur isotope ratio (any one isotope selected from ³³S, ³⁴S, and ³⁶S/³²S isotope) of ³⁴S concentrated internal standard solution (y),

R_{z}: Sulfur isotope ratio (any one isotope selected from ³³S, ³⁴S, and ³⁶S/³²S isotope) of sulfur standard solution (z),

Rₓᵢ: Sulfur i-th isotope ratio (³²S/³²S, ³³S/³²S, ³⁴S/³²S, ³⁶S/³²S) of sample or sample solution (x),

R_{zi}: Sulfur i-th isotope ratio (³²S/³²S, ³³S/³²s, ³⁴S/³²S or ³⁶S/³²S) of sulfur standard solution (z) for calibration,

R_{b}: Sulfur isotope ratio (any one isotope selected from ³³S, ³⁴S, and ³⁶S/³²S isotope) of sample blend (b),

R_{b′}: Sulfur isotope ratio (any one isotope selected from ³³S, ³⁴S, and ³⁶S/³²S isotope) of calibration blend (b)

w: Dry mass calibration factor, and

C_{blank}: Concentration of blank sample.

Sulfur impurities may be present in a reagent and a solvent to be added in a sample pre-treatment process, and in a container, such that a sulfur concentration in pure sample may be obtained by preparing a blank sample to calculate C_{blank} (sulfur concentration of the blank sample) and subtracting C_{blank} from the sulfur concentration measured in the sample.

The solvent used in the present invention may be preferably deionized water, an acid solution diluted by the deionized water, or a buffer solution, and a content of sulfur included in the solvent may be preferably 0.02 mg/kg or less. More preferably, the solvent may include sulfur in a content of 10 ng/kg or less. The sulfur content in the sample determined as above may be used to calculate a content of the organic compound by using a molecular formula of the organic compound. However, the sulfur content in the sample determined as above may include other sulfur-containing impurities in addition to sulfur for analysis, such that measurement traceability in SI unit may be achieved by quantifying the sulfur-containing impurities and subtracting the quantified sulfur-containing impurities from total sulfur content. In the measurement method for the sulfur-containing impurities, CE-ICP/MS or SEC-ICP/MS is preferably used for protein, but is not limited thereto. In the following Example, impurities were quantified by using 50 mM ammonium bicarbonate as an eluent at a flow rate of 1 ml/min on BioSep SEC-3000 column (300×4.6mm), and by using UV and ICP/MS.

Hereinafter, the present invention will be described by Examples and Comparative Examples in more detail. These examples are only for exemplifying the present invention, and it will be obvious to those skilled in the art that the scope of the present invention is not construed to be limited to these examples.

### EXAMPLE 1

### Performance of (First) Quantification for Sulfur Element-Based hGH Protein

Sulfur in an organic compound was completely decomposed into an inorganic form to measure a sulfur content, sulfur-containing impurities were evaluated by SEC-ICP/MS, and a quantification method for an organic compound based on measurement for a sulfur content having SI traceability was performed. Human growth hormone (hGH) has an average molecular weight of 22,125 Da, and includes 3 methionines and 4 cysteines, and accordingly, hGH contains 7 sulfur atoms per one molecule. That is, 1 mole of hGH corresponds to 7 moles of sulfur atoms.

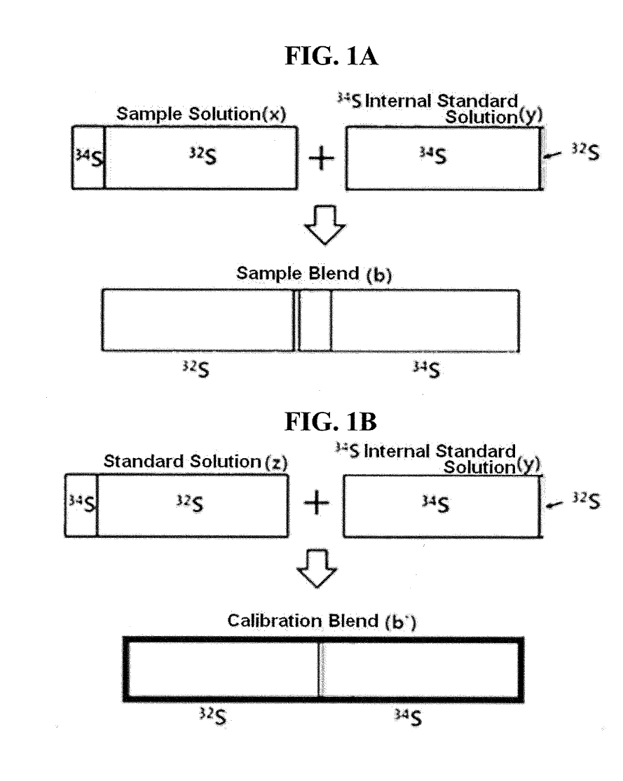

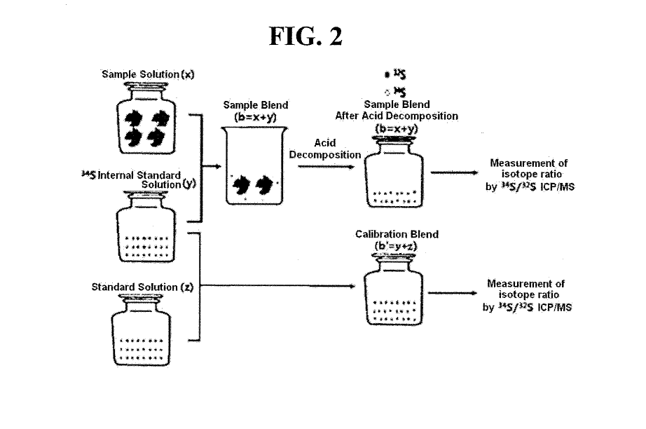

(1) Preparation of Sample Blend and Calibration Blend (see FIGS. 1 and 2)

\textcircled{1} 0.5 g of hGH standard solutions (containing about 65 mg/kg of sulfur content in the first batch) from 5 vials were collected in 5 Teflon series (TFM) vessels, respectively.

\textcircled{2} 32.5 mg/kg of ³⁴S concentrated standard solution (Oak Ridge National Lab) (1 mL) was added to the collected sample to prepare 5 sample blends each having an estimated isotope ratio (³⁴S/³²S) of 1.

\textcircled{3} 32.5 mg/kg of a sulfur standard solution (1 mL) and the ³⁴S concentrated standard solution (1 mL) were mixed and diluted with 30 g of 10% (diluted) HNO₃ to prepare 5 calibration blends each also having an isotope ratio of 1.

\textcircled{4} Nitric acid (3 mL), deionized water (3 mL), and hydrogen peroxide (2 mL) were added to the TFM vessels containing the sample blends.

\textcircled{5} The TFM vessels were sealed and put into an electromagnetic wave oven to heat up to 200° C. for 15 minutes, and maintained at 200° C. for 20 minutes to perform electromagnetic wave-assisted acid decomposition.

\textcircled{6} The TFM vessels were cooled in some degree, and then shaken sufficiently so as to homogenize an inner solution in the vessel.

\textcircled{7} A TFM pressure vessel was disassembled to recover the sample blends and the sample blends were diluted with 30 g of deionized water.

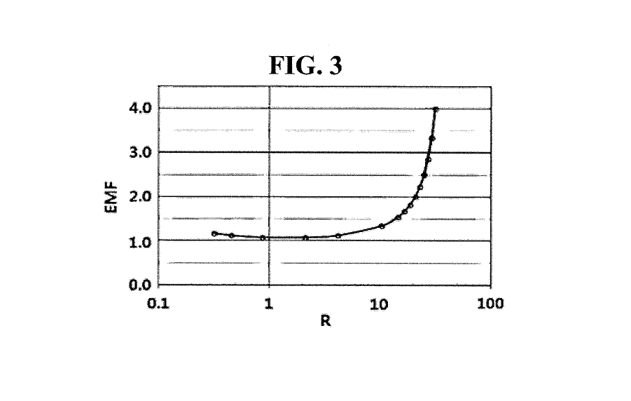

Meanwhile, for an optimum isotope ratio, a measurement deflection is minimized as an isotope ratio (³⁴S/³²S) is close to 1 (see FIG. 3), and therefore, the isotope ratio (³⁴S/³²S) of 1 was determined as a target isotope ratio (³⁴S/³²S) for isotope dilution. Accordingly, in Example 1, a working solution obtained by diluting a sulfur standard solution (SRM 3154) (z) which is a commercially available product prepared by National Institute of Standards and Technology (NIST) was mixed with the ³⁴S concentrated internal standard solution (y) to prepare a calibration blend (b′) having an isotope ratio (³⁴S/³²S) of 1. (see FIGS. 1 and 2).

(2) ICP/MS Measurement

\textcircled{1} Thermo Scientific™ high resolution ICP/MS was optimized to be high resolution (R>10,000). (Medium resolution is sufficient to remove ionization interference produced from acid, solvent, organic material, and ICP gas; however, in order to minimize an effect by high background signal of deionized water, acid, and sulfur present in an equipment, measurement is performed by reducing device sensitivity by high-resolution): The prepared sample blend (b) and the calibration blend (b′) exhibited signal intensity of about 600,000-700,000 cps on ³²S and ³⁴S.

\textcircled{2} An isotope ratio was measured while sweeping ³²S and ³⁴S at a rapid speed, and when the isotope ratio was measured by ID-ICP/MS, an isotope ratio of an isotopic ratio standard, of which isotope ratio is known, were periodically measured together to correct for mass bias and signal drift.

\textcircled{3} Measurement values were calculated by the measured isotope ratios, and uncertainty evaluation results on each factor having an influence on the measurement were synthesized to estimate measurement uncertainty.

(3) Measurement Results for Sulfur Content in hGH First Batch by ID-ICP/MS

In the hGH first batch, a concentration provided by a manufacturer was 303 μmol/kg, and it was confirmed as 290 μmol/kg (65 mg/kg of sulfur content) by amino acid-based quantification.

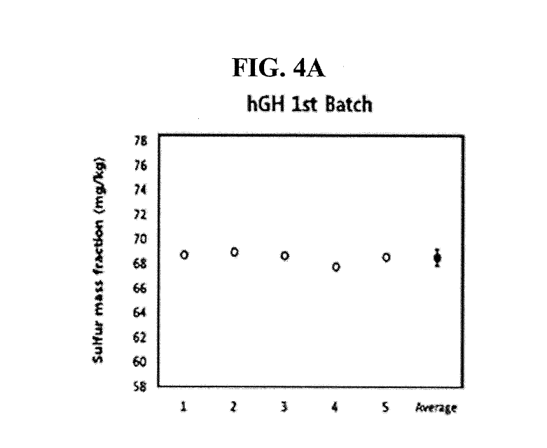

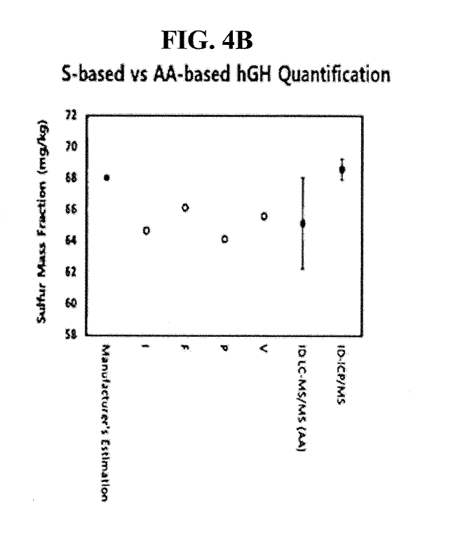

\textcircled{1} Measurement results for sulfur content in hGH first batch were shown in Table 1 and FIG. 4A. As compared to the amino acid-based quantification results according to the related art, the sulfur-based quantification results were 5% higher than that of the amino acid-based quantification results (see FIG. 4B).

TABLE 1
Measurement results for sulfur content in hGH standard
solution in first batch

| | | results | systematic u | |
|---|---|---|---|---|
| | No | (mg/kg) | (mg/kg) | v |
| Sample | 1 | 68.75 | 0.24 | 78 |
| | 2 | 69.00 | 0.24 | 78 |
| | 3 | 68.67 | 0.24 | 78 |
| | 4 | 67.80 | 0.24 | 78 |
| | 5 | 68.59 | 0.24 | 78 |
| Average | | 68.56 | | |
| U_{random} | | 0.20 | | 4 |
| (std of the mean) | | | | |
| U_{systematic pooled} | | 0.24 | | 78 |
| u_{combined} | | 0.32 | | 21 |
| U_{combined} | | 0.66 | | |

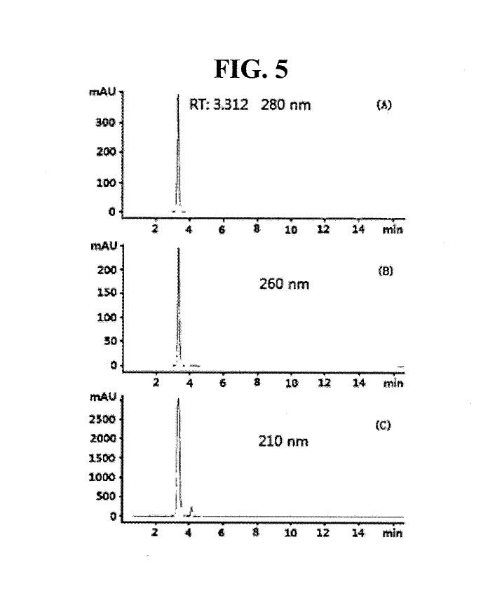

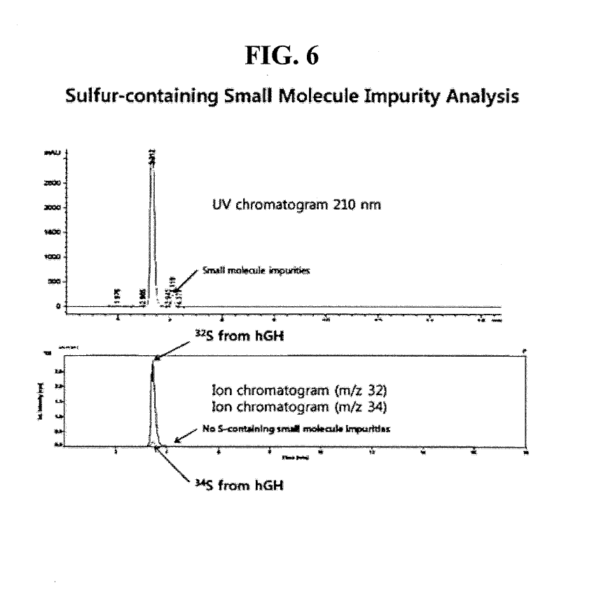

\textcircled{2} In order to confirm the reason that the sulfur element-based protein quantification results were higher than that of the existing amino acid-based quantification results, possibility that sulfur element-containing small molecule impurities in the sample were present to have an influence on total sulfur content was confirmed by size exclusion chromatography (SEC)-UV and SEC-ICP/MS (see FIGS. 5 and 6).

It was confirmed that a small peak eluted after 4 minutes in SEC-UV corresponds to the small molecule, and ion chromatogram only on m/z 32 and m/z 34 corresponding to sulfur ions by SEC-ICP/MS were obtained, such that there were no detectable peaks in corresponding retention time. From the results, an effect of the sulfur-containing small molecule impurities on the measurement for total sulfur content was less than 0.5%. Therefore, it was determined that there is no possibility that element-based protein quantification results were overestimated by the sulfur-containing impurities in Example 1 above.

### EXAMPLE 2

### Performance of (Second Batch) Quantification of Sulfur Element-Based hGH Protein

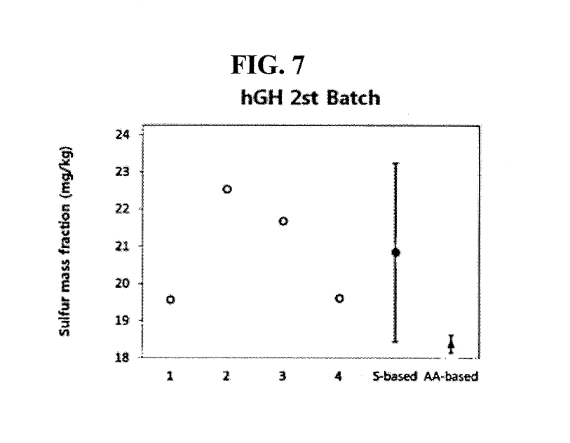

Quantification for hGH protein in hGH second batch which was newly prepared by the same method as Example 1 above was performed. Since a concentration of the sample was about ⅓ times lower than that of the first batch, a collection mass of a sample for analysis was increased from 0.5 g to be 1.0 g, and a concentration of a concentrated isotope solution was controlled accordingly, so that an isotope ratio between the sample blend and the calibration blend was close to be 1 as like Example 1. Since an amount of a sample per a vial of secondarily prepared hGH was not sufficient for a single-dose of the sample, one aliquot sample was collected from 2 or more vials. Analysis results of second batch were shown in FIG. 7 and Table 2.

TABLE 2
Measurement results for sulfur content in hGH standard solution in second
batch

| | | results | systematic u | |
|---|---|---|---|---|
| | No | (mg/kg) | (mg/kg) | v |
| Sample | 1 | 19.56 | 0.07 | 21 |
| | 2 | 22.53 | 0.07 | 21 |
| | 3 | 21.67 | 0.07 | 21 |
| | 4 | 19.60 | 0.07 | 21 |
| Average | | 20.84 | | |
| U_{random} | | 0.75 | | 3 |
| (std of the mean) | | | | |
| U_{systematic pooled} | | 0.07 | | 21 |
| u_{combined} | | 0.75 | | 3 |
| U_{combined} | | 2.40 | | |

### EXAMPLE 3

### Performance of Element-Based Absolute Quantification on hGH T2, T11 Peptides

The same measurement deflection was observed in first and second batches of sulfur element-based hGH quantification. Accordingly, in order to examine possibility that amino acid-based quantification result had been affected by efficiency of hydrolysis and side-reaction, hGH T2 and T11 peptide standard solutions that seem to be easier to perform hydrolysis were prepared, and comparison between amino acid-based quantification results and sulfur element-based quantification results was conducted.

T2 peptide which is a second tryptic peptide from N-terminus of hGH has a sequence of LFDNAMLR wherein methionine containing sulfur is one, such that there is one sulfur atom per one peptide, which is possible to perform sulfur element-based quantification.

T11 peptide also has a sequence of DLEEGIQTLMGR wherein methionine is also one, such that there is one equivalent of sulfur atom per one equivalent of peptide. T2 was manufactured to have a concentration of 1 mmol/kg, a purity thereof provided by a manufacturer was 99.1%, and T11 was manufactured to have a concentration of 1 mmol/kg, a purity thereof provided by a manufacturer was 99%. Accordingly, each estimated content of sulfur elements was 32 mg/kg.

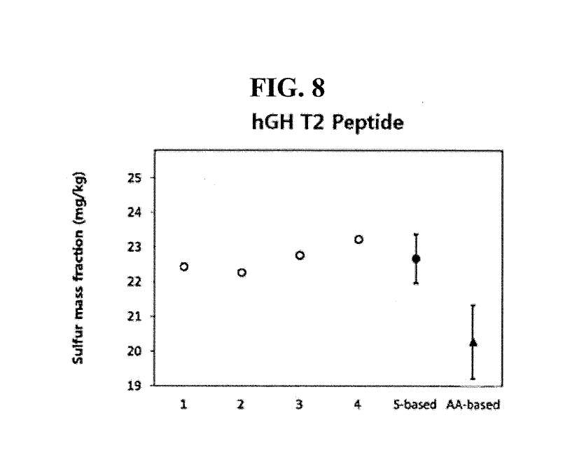

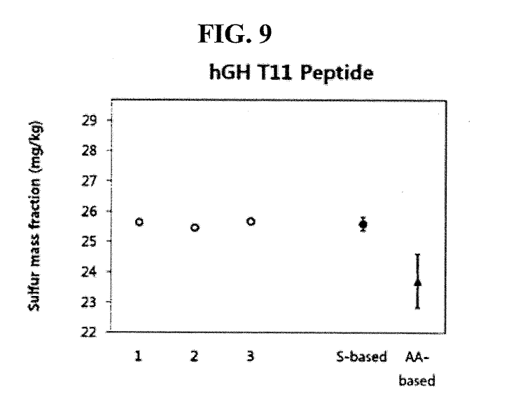

As shown in FIG. 8 and Table 3, a content of T2 peptide could be measured as 3.1% level of expanded uncertainty, through the sulfur element-based peptide quantification. However, in the sulfur element-based quantification results on T2 peptide, similar to the protein quantification, 10% level of measurement deflection was also shown as compared to the amino acid-based quantification. A content of T11 peptide could be quantified as 0.84% of expanded uncertainty; however, measurement deflection similar to T2 peptide was shown (see FIG. 9 and Table 3). Therefore, even during hydrolysis of peptide for amino acid-based quantification, imperfection of hydrolysis and loss due to secondary reaction of acid decomposition are still present or possibility that observed measurement deflection is caused by other factors may not be completely excluded.

TABLE 3
Measurement results for sulfur in hGH T2 peptide standard solution

| | | results | systematic u | |
|---|---|---|---|---|
| | No | (mg/kg) | (mg/kg) | v |
| Sample | 1 | 22.44 | 0.07 | 118 |
| | 2 | 22.27 | 0.07 | 119 |
| | 3 | 22.78 | 0.07 | 117 |
| | 4 | 23.23 | 0.07 | 116 |
| Average | | 22.68 | | |
| U_{random} | | 0.21 | | 3 |
| (std of the mean) | | | | |
| U_{systematic pooled} | | 0.07 | | 117 |
| u_{combined} | | 0.22 | | 3 |
| U_{combined} | | 0.71 | | |

TABLE 4
Measurement results for sulfur in hGH T11 peptide standard solution

| | | results | systematic u | |
|---|---|---|---|---|
| | No | (mg/kg) | (mg/kg) | v |
| Sample | 1 | 22.44 | 0.07 | 118 |
| | 2 | 22.27 | 0.07 | 119 |
| | 3 | 22.78 | 0.07 | 117 |
| | 4 | 23.23 | 0.07 | 116 |
| Average | | 22.68 | | |
| U_{random} | | 0.21 | | 3 |
| (std of the mean) | | | | |
| U_{systematic pooled} | | 0.07 | | 117 |
| u_{combined} | | 0.22 | | 3 |
| U_{combined} | | 0.71 | | |

### EXAMPLE 4

### Performance of Element-Based Absolute Quantification of Methionine

The same measurement deflection as a protein standard solution was observed in a sulfur element-based quantification of the standard solution of the peptide smaller than that of protein, and accordingly, sulfur-containing amino acid standard solutions (Met and Cys) were prepared, and purity analysis was substituted with sulfur element-based quantification assay. Then, the amino acid standard solution was certified to be utilized. The prepared and certified amino acid standard solution could be utilized for amino acid-based qualification of protein and peptide by using LC-MS/MS. Cysteine could be easily changed into disulfide bonds by an oxidation reaction, such that in order to avoid complexity of the analysis, a standard solution of methionine was primarily prepared and an element-based absolute quantification thereof was attempted. Since oxidation, and the like, of methionine easily occurs in the amino acid-based quantification using methionine, at the time of performing LC-MS/MS analysis, oxidized methionine as well as original form of methionine need to be analyzed.

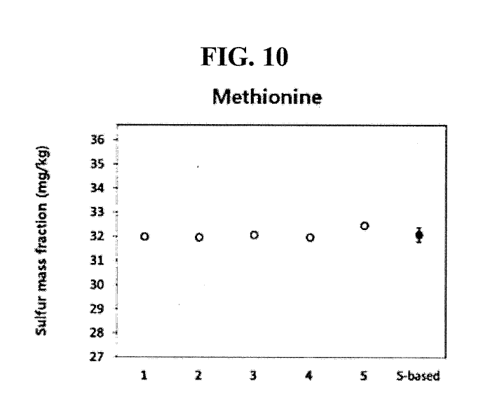

The prepared methionine standard solution had a concentration of 1 mmol/kg, and a purity thereof provided by a manufacturer was 99.5% or more. It corresponds to a sulfur content of 32 mg/kg. Sulfur element-based quantification results of the methionine standard solution were shown in FIG. 10 and Table 5, and quantification results in which expanded uncertainty was within 1% could be obtained. The prepared methionine standard solution may be utilized for amino acid-based quantification of protein including hGH T2, T11 peptides, and methionine in the future, as well as hGH.

TABLE 5
Analysis results of sulfur element-based quantification of methionine
standard solution

| | | results | systematic u | |
|---|---|---|---|---|
| | No | (mg/kg) | (mg/kg) | v |
| Sample | 1 | 31.99 | 0.10 | 348 |
| | 2 | 31.95 | 0.10 | 349 |
| | 3 | 32.07 | 0.10 | 346 |
| | 4 | 31.97 | 0.10 | 348 |
| | 5 | 32.46 | 0.10 | 338 |
| Average | | 32.09 | | |
| U_{random} | | 0.10 | | 4 |
| (std of the mean) | | | | |
| U_{systematic pooled} | | 0.10 | | 345 |
| u_{combined} | | 0.14 | | 15 |
| U_{combined} | | 0.29 | | |

### EXAMPLE 5

### Review of Effectivity on Existing Sample Pre-Treatment Method (Acid Decomposition through Electromagnetic Wave Irradiation) and Small Mmount Sample Pre-Treatment Method

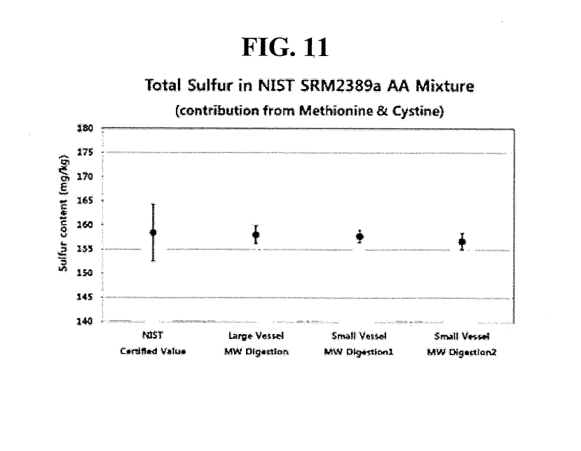

An organic compound was completely decomposed into an inorganic form of sulfur, and electromagnetic wave irradiation and acid decomposition were simultaneously performed in order to obtain sufficient chemical equivalent with the concentrated isotope added as an internal standard solution. The acid decomposition through the electromagnetic wave irradiation used for Examples 1 to 4 is to collect a sample in 100 mL volume of a sealed Teflon vessel, put the vessel into an electromagnetic wave oven, and raise a temperature by heating up to 200° C. for 15 minutes, and maintain a temperature at 200° C. for 20 minutes to perform electromagnetic wave-assisted acid decomposition. Review of effectivity on the used sample pre-treatment method was attempted to be performed by measuring a sulfur content in NIST SRM 2389a (amino acid mixture certified reference material which is certified by general organic compound purity analysis) and comparing the measured sulfur content with the certified value. 0.2 g of the sample was taken into the Teflon vessel, the concentrated isotope ³⁴S(ORNL) diluted to be a pre-calculated concentration was added to the vessel, and 3 mL of 65% nitric acid, 3 mL of deionized water, and 2 mL of 30% hydrogen peroxide were added thereto, to perform acid decomposition. In addition, review of effectivity on the pre-treatment for the small amount sample acid decomposition was performed by analyzing NIST SRM 2389a sample. 0.2 g of the sample was taken into a Teflon vessel for small sample, the concentrated isotope ³⁴S(ORNL) diluted to be a pre-calculated concentration was added to the vessel, and 3 mL of 65% nitric acid was added thereto. The mixture was put into an electromagnetic wave oven to raise a temperature up to 120° C. for 8 minutes, and raising a temperature up to 220° C. for 7 minutes, and then maintaining a temperature at 220° C. for 20 minutes to perform electromagnetic wave-assisted acid decomposition. NIST SRM2 389a sample contains methionine (0.3733±0.0108) mg/kg, and cysteine (0.2954±0.0133) mg/kg, as an amino acid standard solution. Methionine has 1 sulfur atom and cysteine has 2 sulfur atoms, such that the sulfur content contained in the NIST SRM 2389a sample is (158.4±4.2) mg/kg. Measurement results for sulfur content of NIST SRM 2389a sample were shown in Table 6 and FIG. 11. It could be confirmed that sulfur content values measured in both of the sample in which the acid decomposition was performed by the sample pre-treatment method used in Examples 1 to 4 and the sample in which the acid decomposition was performed by the small amount sample pre-treatment method were identical with the certified values of NIST SRM 2389a within the scope of uncertainty.

TABLE 6
Sulfur measurement results of amino acid standard solution
NIST SRM 2389a

| | | Expanded |
|---|---|---|
| NIST SRM2389a | Value, mg/kg | uncertainly, mg/kg |
| Certified value | 158.4 | 4.2 |
| Large Vessel MW Digestion | 158.0 | 1.8 |
| Small Vessel MW Digestion1 | 157.7 | 1.2 |
| Small Vessel MW Digestion2 | 156.7 | 1.7 |

### EXAMPLE 6

### Performance of (Second Batch) of Certification of Sulfur Element-Based hGH Protein

hGH protein certification with respect to newly manufactured hGH second batch was performed.

(1) Preparation of Sample Blend and Calibration Blend

\textcircled{1} For application of isotope dilution mass spectrometry, concentrated isotope ³⁴S concentrated standard solution (Oak Ridge National Lab.) was prepared by dilution to have a calculated concentration of 3.99 mg/kg. 1 g of the prepared dilution was added to 11 Teflon vessels (sample blend: sample blend solution) and 4 LDPE bottles (calibration blend: calibration blend solution), respectively. The concentrated isotope solution was firstly added to 2 bottles for calibration blend solution, then, added to 11 vessels for sample blend solutions, and lastly, added to remaining 2 bottles for calibration blend solution. Here, a ³⁴S concentrated standard solution (Oak Ridge National Lab.) was added to prepare a sample blend so that a measurement-estimated isotope ratio (³⁴S/³²S) is 1.

\textcircled{2} 0.2 g of the samples from 11 hGH standard solution vials were collected into 11 microwave digestion Teflon vessels in which the concentrated isotope solution was added. (11 sample blends)

\textcircled{3} A sulfur standard solution (NIST SRM 3154) used for the calibration blend solution was diluted to have a concentration of 4.12 mg/kg, and 1 g thereof was added to 4 LDPE bottles prepared in \textcircled{1} in which 1 g of ³⁴S concentrated isotope solution was added, and mixed well. The obtained mixture was diluted with 30 g of 5% nitric acid, thereby preparing 4 calibration blend solutions so that an estimated isotope ratio is 1.

\textcircled{4} 3 mL of 65% sub-boiled nitric acid was added to the vessels with the sample blends.

\textcircled{5} MW-assisted acid decomposition was performed by covering the TFM vessel and increasing a temperature up to 120° C. for 8 minutes by heating in UltraWAVE Microwave Digestion System (manufactured from Milestone), and then increasing the temperature up to 220° C. for 7 minutes, and maintaining at 220° C. for 20 minutes.

\textcircled{6} After the acid decomposition, the vessels were allowed to be cooled in some degree, and the sample blends were recovered and diluted with 30 g.

(2) ICP/MS Measurement

\textcircled{1} Agilent 8800 ICP-Triple Quad (ICP-QQQ) was optimized by O2 mode. (With respect to ³²S and ³⁴S, filtering was performed on m/z=32, 34 at quadrupole of 1, followed by reaction with O₂ gas, and filtering on m/z=48 and 50 which are ³²S¹⁶O+ and ³⁴S¹⁶O+ at quadrupole of 2 to perform detection)

\textcircled{2} 20 mL of 5% nitric acid was added to the recovered sample (diluted 17 times), and an isotope ratio was measured. (about 250,000 counts of signal intensity were shown on ³²S and ³⁴S) When the isotope ratio was measured, an isotope ratio of an isotopic ratio standard, of which isotope ratio is known, were periodically measured together to correct for mass bias and signal drift.

\textcircled{3} The above-described measurements were repeatedly performed by three times on the sample, and measurement uncertainty was estimated by using the measured isotope ratios to calculate values, and synthesizing uncertainty evaluation results on each factor having an effect on the measurement.

(3) Certified Results of Sulfur Content in hGH Second Batch by ID-ICP/MS

In the hGH second batch, a concentration provided by a manufacturer was 2 mg/mL, an amino acid-based quantification value corresponds to 1.8 mg/mL, and a sulfur content corresponds to 18.2 mg/kg.

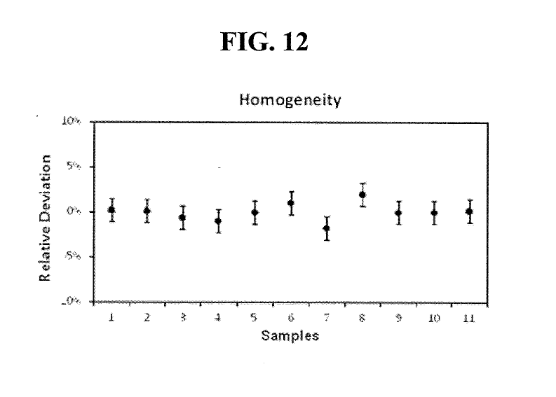

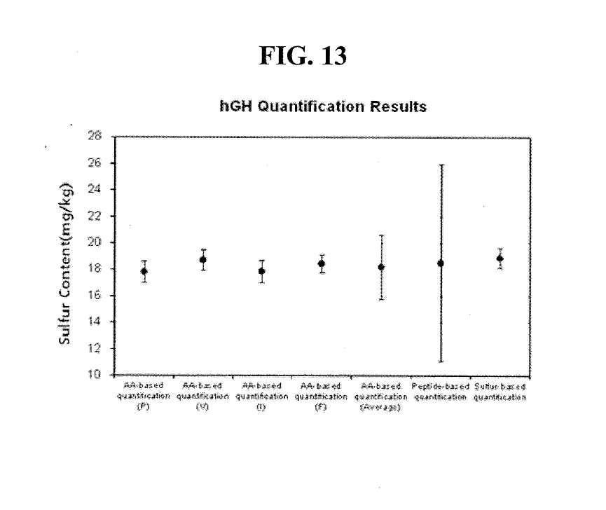

\textcircled{1} A reference value of measurement results for a sulfur content of the hGH second batch is an average value of the measurement results of samples collected in 11 vials, and homogeneity was estimated from standard deviation of measurement values on each of 11 samples. With respect to measurement uncertainty, combined standard uncertainty and degree of freedom were calculated from an average value of standard deviations measured from 11 samples and pooled standard deviation value mainly including uncertainty due to a systematic effect in measurement of a sample, and then, a coverage factor and expanded uncertainty were calculated therefrom. As shown in Table 7 and FIG. 12, the standard deviation of the measurement values on each vial was 0.98% of the average value, which may be appreciated that homogeneity among vials is significantly excellent. Accordingly, it may be appreciated that homogeneity appropriate for being usable as a certified reference material is proved. Sulfur content certified values and expanded uncertainty in the hGH second batch were (18.88±0.75) mg/kg as shown in Table 7. As compared to the amino acid- and peptide-based quantification results, it could be confirmed that the certified values are identical within a scope of uncertainty. (See FIG. 13)

TABLE 7
Sulfur measurement-based certified results in hGH standard solution in
second batch

| | | | Standard uncertainly | Degree of |
|---|---|---|---|---|
| | | Analytical results | due to the systematic | freedom |
| | No | (mg/kg) | effects (mg/kg) | (?) |
| Sample | 1 | 18.92 | 0.24 | 3 |
| | 2 | 18.90 | 0.24 | 3 |
| | 3 | 18.77 | 0.24 | 3 |
| | 4 | 18.70 | 0.24 | 3 |
| | 5 | 18.87 | 0.24 | 3 |
| | 6 | 19.08 | 0.24 | 3 |
| | 7 | 18.55 | 0.24 | 3 |
| | 8 | 19.25 | 0.24 | 3 |
| | 9 | 18.88 | 0.24 | 3 |
| | 10 | 18.88 | 0.24 | 3 |
| | 11 | 18.91 | 0.24 | 3 |

| Pooled standard deviation and | | 0.24 | 3 |
|---|---|---|---|
| degree of freedom due | | | |
| to the systematic effects | | | |
| Mean | 18.88 | | |
| Standard | 0.18 | | 10 |
| deviation | | | |
| Relative | 0.98% | | |
| standard | | | |
| deviation | | | |

| | |
|---|---|
| Certified value | 18.88 |
| Combined | 0.30 |
| standard | |
| uncertainly (u_{c}) | |
| Degree of | 6 |
| freedom | |
| Coverage | 2.45 |
| factor (k) | |
| Expanded | 0.75 |
| Uncertainty | |
| (U) at | |
| 95% level | |
| of | |
| confidence | |
| Relative | 3.95% |
| Exp. | |
| Uncertainly | |

1. A quantification method for a sulfur-containing organic compound, using a sulfur isotope ratio.

2. The quantification method of claim 1, wherein the quantification method includes steps (1) to (4) below:
(1) preparing a sample blend so that a theoretical isotopic ratio between any one isotope selected from ³³S, ³⁴S, and ³⁶S and a base isotope (³²S) having the largest natural abundance ratio is 0.2 to 5, by diluting a sample solution including the sulfur-containing organic compound with an internal standard solution in ch any one isotope selected from ³³S, ³⁴S, and ³⁶S is concentrated;
(2) preparing a calibration blend so as to have a theoretical isotopic ratio similar to that of the sample blend prepared in step (1), by mixing the internal standard solution in which one isotope selected from ³³S, ³⁴S, and ³⁶S is concentrated with a sulfur standard solution having a natural abundance ratio;
(3) recovering an inorganic form of sulfate, by decomposing the sulfur-containing organic compound from the sample blend prepared in step (1); and
(4) measuring an isotope ratio between any one isotope selected from ³³S, ³⁴S, and ³⁶S of the sulfate recovered in step (3) and ³²S, and measuring an isotope ratio between a one isotope selected from ³³S, ³⁴S, and ³⁶S of the calibration blend prepared in step (2) and ³²S.

3. The quantification method of claim 1, wherein the sulfur-containing organic compound is protein or peptide including methionine, cysteine, or both of methionine and cysteine.

4. The quantification method of claim 2, wherein in step 3), the sample blend including the sulfur-containing organic compound is treated by simultaneously performing electromagnetic wave irradiation and acid decomposition to be decomposed into the inorganic form of sulfate.

5. The quantification method of claim 4, wherein the simultaneously performing of the electromagnetic wave irradiation and the acid decomposition is conducted by adding an oxidizing agent to the sulfur-containing organic compound, followed by the electromagnetic wave irradiation at 100 to 300° C. temperature to perform the acid decomposition.

6. The quantification method of claim 5, wherein the oxidizing agent is nitric acid, perchloric acid, hydrogen peroxide, or a mixture thereof.

7. The quantification method of claim 2, wherein the isotope ratio between any one isotope selected from ³³S, ³⁴S, and ³⁶S of the sample blend and ³²S, and the isotope ratio between any one isotope selected from ³³S, ³⁴S, and ³⁶S of the calibration blend and ³²S, obtained in step (4) are applied to Equation 1 below:
$${C}_{x} = \{ {C}_{z} \cdot \frac{{m}_{z} {m}_{y}}{{wm}_{z} {m}_{y}} \cdot \frac{{R}_{y} - {R}_{b}}{{R}_{b} - {R}_{x}} \cdot \frac{{R}_{b} - {R}_{z}}{{R}_{y} - {R}_{b}} \cdot \frac{{\sum }_{i} {R}_{xi}}{{\sum }_{i} {R}_{zi}} \} - ( {C}_{blank} ) \qquad 〈 Equation 1 〉$$
Cₓ: concentration of sample solution (x) to be measured,
mₓ: Mass of sample solution (x) measured by using an analytical balance,
m_{z}: Mass of sulfur standard solution (z) measured by using an analytical balance,
m_{y}: Mass of internal standard solution (y) in which any one isotope selected from ³³S, ³⁴S, and ³⁶S added to sample solution (x) is concentrated,
m_{y′}: Mass of internal standard solution (y) in which any one isotope selected from ³³S, ³⁴S, and ³⁶S added to sulfur standard solution (z) for calibration is concentrated,
C_{z}: Concentration of sulfur standard solution (z) for calibration,
Rₓ: Sulfur isotope ratio (any one isotope selected from ³³S, ³⁴S, and ³⁶S/³²S isotope) of sample solution (x),
R_{y}: Sulfur isotope ratio (any one isotope selected from ³³S, ³⁴S, and ³⁶S/³²S isotope) of an internal standard solution (y) in which any one isotope selected from ³³S, ³⁴S, and ³⁶S is concentrated,
R_{z}: Sulfur isotope ratio (any one isotope selected from ³³S, ³⁴S, and ³⁶S/³²S isotope) of sulfur standard solution (z),
Rₓᵢ: Sulfur i-th isotope ratio (³²S/³²S, ₃₃S/³²S, ³⁴s/³²S, ³⁶S/³²S)of sample solution (x),
R_{zi}: Sulfur i-th isotope ratio (³²S/³²S, ₃₃S/³²S, ³⁴S/³²S or ³⁶S/³²S) of sulfur standard solution (z) for calibration,
R_{b}: Sulfur isotope ratio (any one isotope selected from ³³S, ³⁴S, and ³⁶S/³²S isotope) of sample blend,
R_{b′}: Sulfur isotope ratio (any one isotope selected from ³³S, ³⁴S, and ³⁶S/³²S isotope) of calibration blend,
w: Dry mass calibration factor, and
C_{blank}: Concentration of blank sample.
